(19) United States
(12) Patent Application Publication
(10) Pub. No.: US 2024/0060992 A1
(43) Pub. Date: Feb. 22, 2024

(54) METHOD FOR PREDICTING CAUSE-SPECIFIC THERAPEUTIC EFFECT OF SENSORINEURAL HEARING LOSS, AND DIAGNOSTIC KIT USED THEREFOR

(71) Applicants: Seoul National University Hospital, Seoul (KR); The Industry & Academic Cooperation in Chungnam National University (IAC), Daejeon (KR)

(72) Inventors: Byung Yoon Choi, Yongin-si (KR); Bong Jik Kim, Seoul (KR)

(73) Assignees: Seoul National University Hospital, Seoul (KR); The Industry & Academic Cooperation in Chungnam National University (IAC), Daejeon (KR)

(21) Appl. No.: 18/385,354

(22) Filed: Oct. 30, 2023

Related U.S. Application Data
(63) Continuation-in-part of application No. PCT/KR21/13682, filed on Oct. 6, 2021.

(30) Foreign Application Priority Data
Apr. 30, 2021 (KR) 10-2021-0056601
Jul. 21, 2021 (KR) 10-2021-0095924

(51) Int. Cl. G01N 33/68

(52) U.S. Cl. CPC G01N 33/6893; G01N 2333/545; G01N 2800/14; G01N 2800/52

(57) ABSTRACT

The present disclosure relates to a kit and an information providing method capable of predicting or diagnosing a cause-specific therapeutic effect of sensorineural hearing loss. The kit and the method according to one aspect of the present invention can diagnose a cause-specific therapeutic effect of sensorineural hearing loss at an early stage and allow selection of the most appropriate treatment method.

### TECHNICAL FIELD

The present disclosure discloses an information providing method for prediction or diagnosis of a therapeutic effect based on etiology of sensorineural hearing loss and a diagnostic kit used therein.

### DESCRIPTION OF THE RELATED ART

Gradually progressive sensorineural hearing loss (SNHL) is a widespread sensory defect. Ranging from acute sensorineural hearing loss that worsens within 2-3 days of onset to subacute worsening over a longer period of time, often months, SNHL is a syndrome rather than a single disease due to its diverse etiology.

Because the cause of sensorineural hearing loss is difficult to identify, steroids are commonly used systemically or topically. However, steroid treatments that are not cause-specific are often ineffective and are limited by resistance to steroid therapy itself.

Given this background, the inventor of the present disclosure reviewed the various pathogenesis of sensorineural hearing loss and studied potential biomarkers of sensorineural hearing loss progression and predictors for efficient treatment to complete the present disclosure.

### DISCLOSURE OF THE INVENTION

According to one aspect, the present disclosure aims to provide an information providing method for prediction or diagnosis of a therapeutic effect based on etiology of sensorineural hearing loss.

According to another aspect, the present disclosure aims to provide a kit for prediction or diagnosis of a therapeutic effect based on etiology of sensorineural hearing loss.

According to one aspect, the present disclosure provides an information providing method for prediction or diagnosis of therapeutic effect based on etiology of sensorineural hearing loss, including (a) inducing IL-1β secretion from a sample isolated from a subject, (b) measuring the IL-1β secretion induced in step (a), and (c) comparing the IL-1β secretion measured in step (b) to a sample of a normal subject.

According to another aspect, the present disclosure provides a kit for prediction or diagnosis of therapeutic effect based on etiology of sensorineural hearing loss in a subject including an IL-1β secretion-inducing agent and an IL-1β secretion-measuring agent.

According to one aspect, the method or kit according to an embodiment of the present disclosure is effective in predicting or diagnosing the therapeutic effect based on etiology of sensorineural hearing loss.

According to one aspect, the method or kit according to an embodiment of the present disclosure may be used clinically to determine treatment by diagnosing the therapeutic effect based on etiology of sensorineural hearing loss early on and selecting the most appropriate treatment method.

### BRIEF DESCRIPTION OF THE DRAWINGS

FIG. 1 is a table depicting genotypic and phenotypic characteristics of a subject according to an embodiment of the present disclosure.

FIGS. 2A to 2M are graphs depicting hearing thresholds in relation to blood levels of inflammatory markers (ESR/CRP) in subjects according to an embodiment of the present disclosure.

FIG. 3 is a graph depicting IL-1β secretion levels in a subject according to an embodiment of the present disclosure.

### DETAILED DESCRIPTION OF THE INVENTION

The present disclosure is described in detail below.

According to one aspect, the present disclosure provides an information providing method for prediction or diagnosis of therapeutic effect based on etiology of sensorineural hearing loss, including (a) inducing IL-1β secretion from a sample isolated from a subject, (b) measuring the IL-1β secretion induced in step (a), and (c) comparing the IL-1β secretion measured in step (b) to a sample of a normal subject. According to an aspect of the present disclosure, “prediction or diagnosis of therapeutic effect based on etiology of sensorineural hearing loss” may mean predicting or diagnosing the etiology of a subject's sensorineural hearing loss or whether a subjects sensorineural hearing loss will respond to treatment with an IL-1β antagonist. The method or kit according to an aspect of the present disclosure may be used clinically to determine treatment by diagnosing the therapeutic effect based on etiology of sensorineural hearing loss early on and selecting the most appropriate treatment method.

According to one aspect, the subject may be a sensorineural hearing loss patient who has an oversecretion of IL-1β compared to a normal subject.

According to one aspect, if the subject's hearing level before administration of an IL-1β antagonist is 60 dB or less, the hearing level after administration of the IL-1β antagonist may be predicted to be no worse than 60 dB.

According to one aspect, step (a) may induce the IL-1β secretion with one or more agents selected from a group consisting of LPS, ATP, and CaCl₂).

According to one aspect, the sample in step (a) may be whole blood, peripheral blood mononuclear cells (PBMCs) derived from whole blood, serum, or saliva.

According to one aspect, the measurement of IL-1β secretion in step (b) may be performed by a rapid test kit such as enzyme-linked immunosorbent assay (ELISA), RT-PCR, or rapid antigen test. Here, measurement of IL-1β secretion includes qualitative measurement of whether IL-1β is secreted or not, or quantitative measurement of the level of IL-1β secretion.

According to another aspect, the present disclosure provides a kit for prediction or diagnosis of therapeutic effect based on etiology of sensorineural hearing loss in a subject comprising an IL-1β secretion-inducing agent and an IL-1β secretion-measuring agent.

According to an embodiment, the subject may be a sensorineural hearing loss patient who has an oversecretion of IL-1β compared to a normal subject.

According to an embodiment, if the subject's hearing level before administration of an IL-1β antagonist is 60 dB or less, the hearing level after administration of the IL-1β antagonist may be predicted to be no worse than 60 dB.

According to an embodiment, the IL-1β secretion-inducing agent may include one or more agents selected from a group consisting of LPS, ATP, and CaCl₂).

According to an embodiment, the IL-1β secretion-measuring agent may include one or more agents selected from a group consisting of a primer, a probe, and an antibody.

As used herein, “primer” means a polynucleotide or variant thereof having bases of a sequence capable of binding complementarily to the terminals of a specific region of a gene used to amplify by PCR a specific region corresponding to the target site of the gene. The primers are not required to be completely complementary to the terminal of the specific region, and may be used as long as they are complementary enough to hybridize to the terminal and form a double-chain structure.

As used herein, “probe” means a polynucleotide, a variant thereof, or a polynucleotide and a labeling agent conjugated thereto having bases of a sequence capable of binding complementarily to a target site of a gene.

As used herein, “hybridization” means that two single-stranded nucleic acids form a duplex structure by pairing of complementary base sequences. Hybridization may occur with perfect complementarity between single-stranded nucleic acid sequences, as well as with the presence of some mismatched bases.

According to an embodiment, the antibody may be one or more selected from the group consisting of polyclonal antibodies, monoclonal antibodies, recombinant antibodies, and combinations thereof. Specifically, the antibody may include polyclonal antibodies, monoclonal antibodies, recombinant antibodies, and both complete forms having two full-length light chains and two full-length heavy chains, as well as functional fragments of antibody molecules, such as Fab, F(ab), F(ab′)2, and Fv. Antibody production may be readily accomplished using techniques well known in the art to which the present disclosure belongs, and manufactured and commercially available antibodies may be used.

According to an embodiment, a kit according to an aspect of the present disclosure may further comprise, in addition to the IL-1β secretion-inducing agent and the IL-1β secretion-measuring agent, labels enabling quantitative or qualitative measurement of the formation of antigen-antibody complexes, and conventional tools, reagents, and the like used in immunological assays.

According to an embodiment, the labels that enable qualitative or quantitative measurement of the formation of antigen-antibody complexes include enzymes, fluorophores, ligands, luminescents, microparticles, redox molecules, and radioisotopes, but are not necessarily limited thereto. Enzymes that may be used as detection labels include β-glucuronidase, β-glucosidase, β-galactosidase, urease, peroxidase, alkaline phosphatase, acetylcholinesterase, glucose oxidase, hexokinase, GDPase, and RNase, glucose oxidase, luciferase, phosphofructokinase, phosphoenolpyruvate carboxylase, aspartate aminotransferase, phosphoenolpyruvate decarboxylase, and β-latamase, but are not limited thereto. Fluorophores include, but are not limited to, fluorescein, isothiocyanate, rhodamine, phycoerythrine, phycocyanin, allophycocyanin, o-phthaldehyde, and fluorescein. Ligands include, but are not limited to, biotin derivatives. Luminescent agents include, but are not limited to, acridinium esters, luciferin, and luciferase. Microparticles include, but are not limited to, colloidal gold and colored latex. Redox molecules include, but are not limited to, ferrocene, ruthenium complexes, biogenes, quinones, Ti ions, Cs ions, diimides, 1,4-benzoquinones, hydroquinones, K₄ W(CN)⁸, [Os(bpy)₃]²⁺, [RU(bpy)₃]²⁺, and [MO(CN)₈]⁴⁻. Radioisotopes include, but are not limited to, ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³H, and ¹⁸⁶Re.

According to an embodiment, examples of such tools or reagents include, but are not limited to, suitable carriers, solvents, detergents, buffers, stabilizers, and the like. If the label is an enzyme, it may include a substrate and a reaction stopper to measure the enzyme activity. Carriers may be soluble or insoluble, with examples of soluble carriers being physiologically acceptable buffers known in the art, such as PBS, and examples of insoluble carriers being polystyrene, polyethylene, polypropylene, polyester, polyacrylonitrile, fluoropolymers, crosslinked dextran, polysaccharides, other papers, glass, metals, agarose, and combinations thereof.

According to an embodiment, the kit may include a test part that induces IL-1β secretion from a sample isolated from a subject, a measurement part that measures the induced IL-1β secretion, and an analyzer that compares the measured IL-1β secretion to a sample of a normal subject.

According to an embodiment, the test part may, for example, induce IL-1β secretion from a sample with an IL-1β secretion-inducing agent such as LPS, ATP, and CaCl₂). Here, the sample may be whole blood, PBMCs derived from whole blood, serum, or saliva.

According to an embodiment, the kit may further include a PBMC isolation extractor.

According to an embodiment, the measurement part may, for example, measure IL-1β secretion through ELISA, RT-PCR, or rapid antigen test.

According to an embodiment, the kit may further include a display that displays a difference or percentage change in IL-1β secretion before and after inducing IL-1β secretion.

According to an embodiment, the kit may further include an autoimmune disease or fluorescent treponemal antibody absorption test (FTA-ABS) testing unit. The autoimmune diseases include, but are not limited to, Wegener's granulomatosis with polyangiitis, Cogan's syndrome, and nodular polyarteritis nodosa.

Hereinafter, the structure and effect of the present disclosure will be described in more detail with reference to the examples. However, the following examples are provided only for the purpose of illustration in order to help the understanding of the present disclosure, and the scope and range of the present disclosure is not limited thereto.

### WORKING EXAMPLES

### [Example 1] Subject Selection

Seventeen patients with clinically diagnosed cryopyrin-associated periodic syndrome (CAPS) and two patients classified as autoinflammatory type hearing loss (AIHL) were registered as subjects of a study according to an embodiment of the present disclosure. The subjects were examined and their symptoms characterized to assess whether they had CINCA (chronic infantile, neurological, cutaneous and articular) syndrome, MWS (Muckle-Wells syndrome), FCAS (familial cold autoinflammatory syndrome) or DFNA34 (non-syndromic SNHL). In addition, two ‘seemingly AIHL’ patients with no family history were enrolled for comparison with CAPS patients.

Written informed consent was obtained from all subjects, and in the case of minors, written informed consent was obtained from parents or guardians. All steps in this study were approved by the Institutional Review Boards of Seoul National University Hospital and Seoul National University Bundang Hospital.

### [Example 2] Diagnosis of Therapeutic Effect Based on Etiology of Sensorineural Hearing Loss in Subjects

Clinical data including gender, age, medical history, physical examination, and audiological test results were obtained for the subjects selected in Example 1. The hearing threshold was calculated by averaging the thresholds of 0.5, 1, 2, and 4 kHz, and hearing level was classified into four categories: mild (26-40 dB), moderate (41-55 dB), moderately severe (56-70 dB), severe (71-90 dB), and profound (>90 dB). The specific methods used to obtain the clinical data were as follows.

Molecular Genetic Diagnosis

Genomic DNA was extracted from the peripheral blood or buccal swab of the subjects per manufacturer's protocol. Then, whole NLRP3 gene was screened to identify the causative variant. If the potential candidate variant was identified through NLRP3 screening, segregation study was performed to confirm the genetic diagnosis. In cases with no detection of any potentially pathogenic NLRP3 variant, exome sequencing was done to investigate other possible candidate genes, which was followed by the filtering process using bioinformatics analysis.

Clinical Evaluation: Audiologic and Radiologic Data Review

Physical examination was done to document the clinical characteristics and to diagnose CAPS by two experienced pediatric rheumatologists and two otologists. Audiologic evaluation was performed depending on test eligibility (age-dependent): Pure-tone audiometry and/or Auditory brainstem response, and/or Auditory steady-state response. Internal auditory canal protocol MRI including FLAIR sequence was performed to assess whether tumorous condition or inflammation existed in cerebellopontine angle, internal auditory canal or cochlea.

Statistical Analysis

Statistical analysis was performed using Prism v.8.0 software (GraphPad Software, Inc. San Diego, CA, USA) and Statistics v.24 (IBM, Armonk, NY, USA) for Windows. Fisher's exact test was used to determine the association between cochlear enhancement on Brain MRI and hearing outcome. Kruskal-Wallis test was used to compare the IL-1β secretion in response to LPS and LPS+CaCl₂) treatment depending on each individual's medical situation (normal control, DFNA34, AIHL), and Bonferroni adjustment was conducted for post hoc test. P<0.05 was considered statistically significant.

Genotypic Characteristics of Patients with Autoinflammatory Hearing Loss

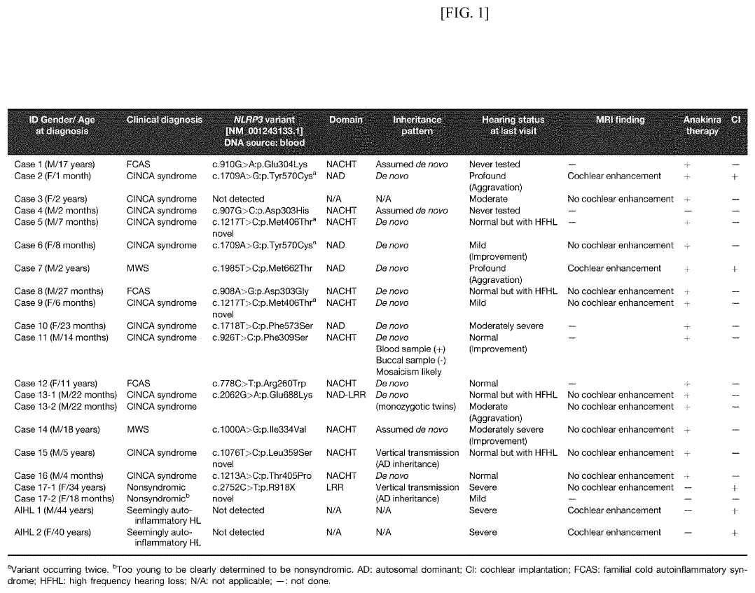

Genotypic and phenotypic characteristics of all subjects with clinically diagnosed CAPS or DFNA34 are shown in FIG. 1. Among the 19 subjects, genetic diagnosis was made in 18 subjects (94.7%), in whom three novel variants of NLRP3 were found with one variant occurring twice in two genetically unrelated individuals (c.1217T>C, Cases 5 and 9). Another variant, c.1709A>G, was also detected in two unrelated individuals (Cases 2 and 6). Autosomal dominant inheritance of the NLRP3 variant from mother to child was shown from two different families (Cases 15 and 17): one with CINCA syndrome (Case 15) and the other with non-syndromic feature (DFNA34) (Case 17).

Audiological Phenotype as Potential Biomarker Predicting the Disease Severity and Treatment Response

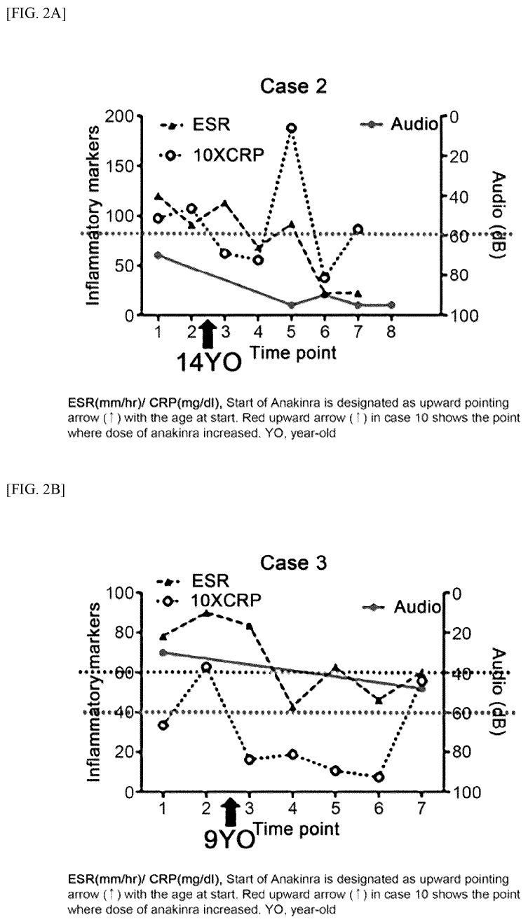

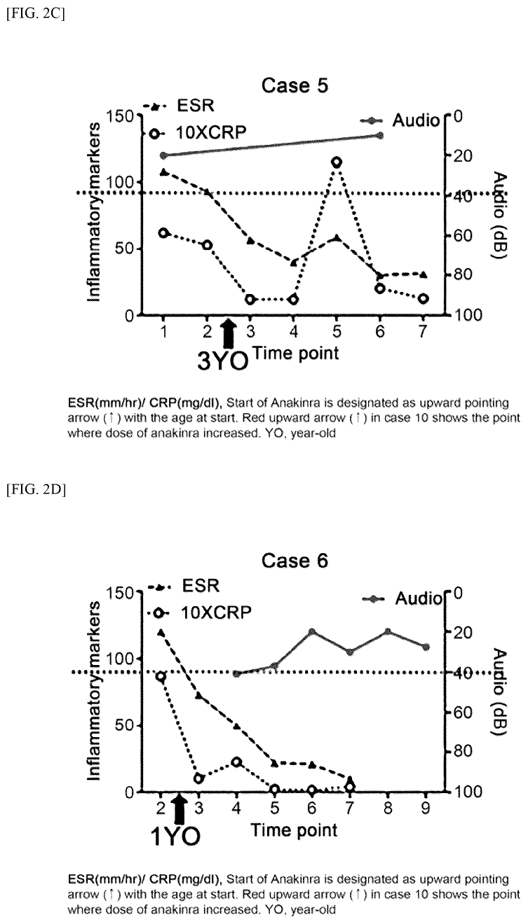

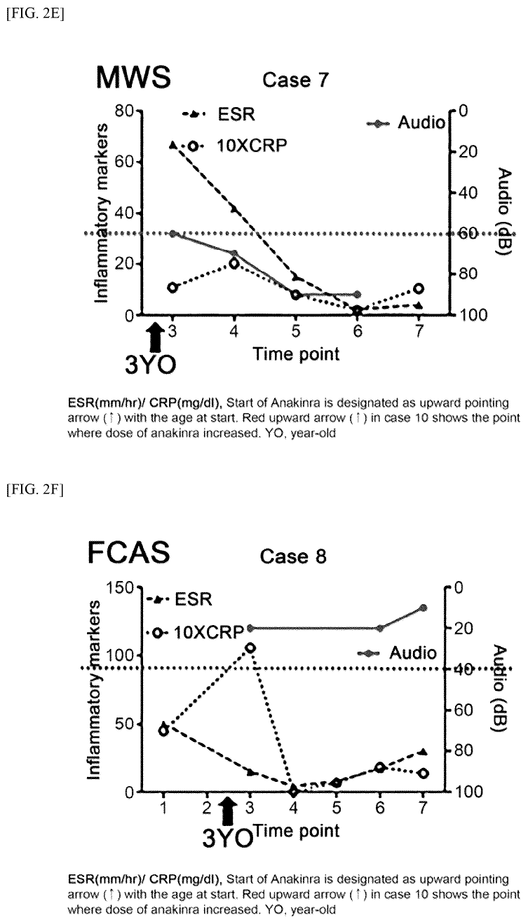

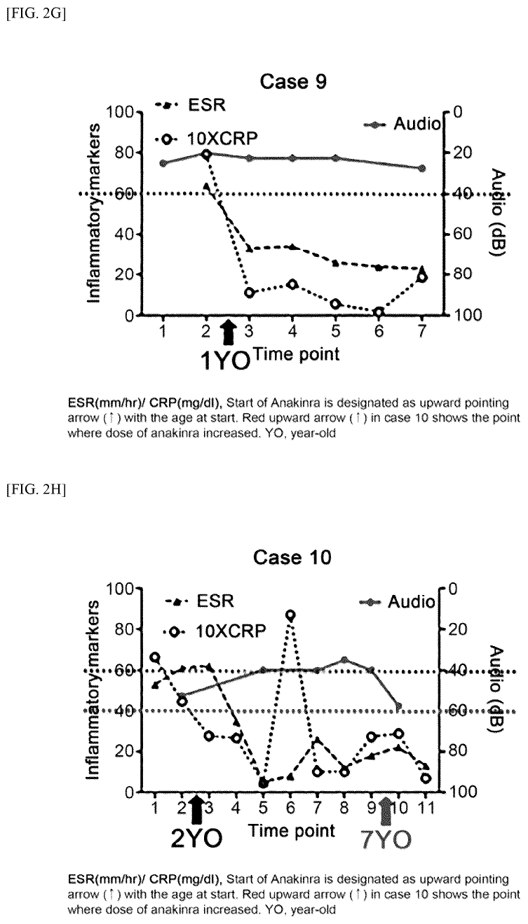

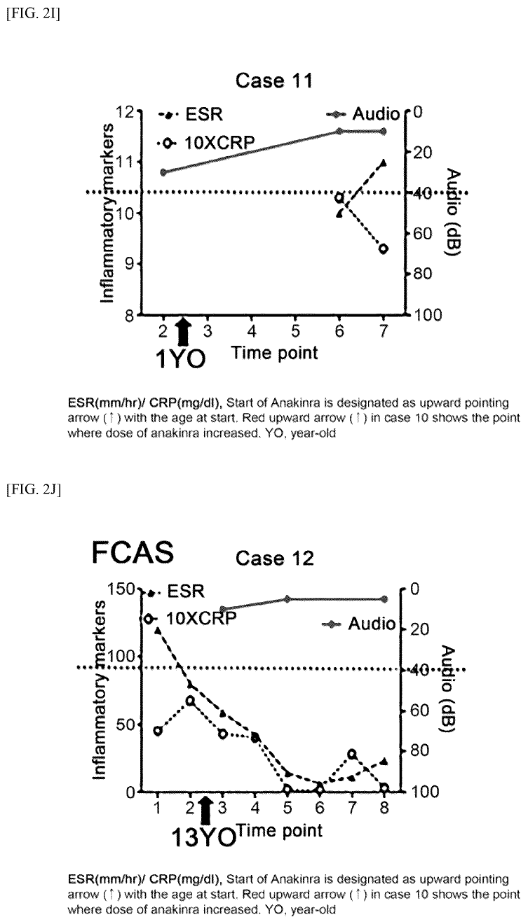

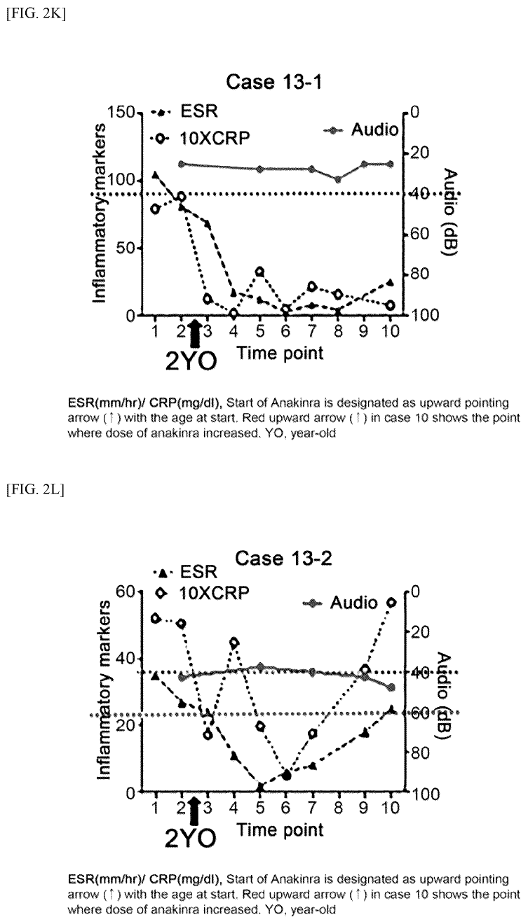

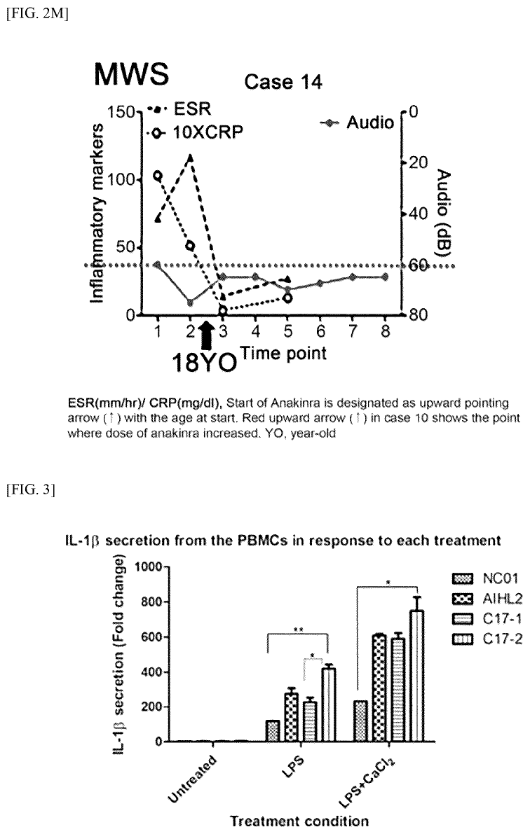

Among 19 subjects, two (Cases 1 (FCAS) and 4 (CINCA syndrome)) have never been tested for hearing and seven (Cases 5, 8, 11, 12, 13-1, 15, and 16) had overall normal hearing thresholds, while four (Cases 5, 8, 13-1, and 15) of the seven subjects had mild hearing loss limited to only high frequency, which might suggest that high frequency hearing is most vulnerable in CAPS patients. Interestingly, four subjects (Cases 3, 5, 6, and 13-2) showed asymmetric hearing loss (a difference of >15 dB between the right and left ears). Audiological phenotype in relation to inflammatory markers was analyzed in the 13 patients with available audiograms and lab data (Cases 2-3, Cases 5-14). Changes in hearing threshold and inflammatory markers, including ESR and CRP, were plotted on time domain (clinic visits) focusing on the use of the IL-1β antagonist anakinra to examine the role of hearing threshold as a potential biomarker of disease progression and responsiveness to anti-IL1 therapy (FIGS. 2A-2M). Contrary to prompt and consistent response of inflammatory markers to the therapy, hearing thresholds showed a differential response to anakinra. In detail, seven genetically confirmed NLRP3-related syndromic patients (Cases 5, 6, 8, 9, 11, 12. and 13-1) starting initially with normal or mild hearing loss, demonstrated stable or slightly improved hearing status in response to anakinra therapy. Additionally, one MWS patient (Case 14) with initially severe SNHL, showed gradual hearing improvement despite delayed anakinra therapy. In summary, hearing status has apparently improved by one level in response to anakinra in three subjects with NLRP3-related CAPS, from mild hearing loss to normal (Cases 11), from moderate to mild hearing loss (Case 6) and from severe to moderately severe hearing loss (Case 14).

On the contrary, hearing thresholds from one CINCA syndrome patient (Case 2) and MWS patient (Case 7) which initially within the range of moderately severe hearing loss eventually progressed to profound hearing loss despite anakinra therapy, and ended up requiring CI. Another patient (Case 3) with obvious CINCA syndrome manifestations but without any definite NLRP3 pathogenic variant, initially demonstrated mild hearing loss that later progressed to moderate hearing loss despite continued anakinra therapy. More intriguingly, two monozygotic twins (Cases 13-1 and 13-2) with CINCA syndrome caused by the same NLRP3 variant, manifested different audiologic phenotypes and divergent responses to anakinra therapy. In detail, Case 13-2 showed gradual aggravation of hearing on one side even with anakinra therapy contrary to Case 13-1 whose hearing status was stable throughout the follow-up period.

Furthermore, as shown in FIGS. 2A-2M, based on a hearing level of 60 dB, it was found that upon administration of anakinra (bold arrow facing up 1) when the hearing level was 60 dB or less, the hearing level was not worse than 60 dB after administration of anakinra (Cases 3, 5, 6, 8, 9, 10, 11, 12, 13-1, 13-2). On the other hand, if the hearing level was above 60 dB, it was found that the hearing level did not improve to better than 60 dB (i.e. to less than 60 dB) after administration of anakinra (Cases 2, 7, 14).

ELISA Assay of IL-1β in Cultured PBMCs

PBMCs were collected from peripheral venous blood samples from four individuals [normal venous 1 (NC01), Cases 17-1 and 17-2, AIHL 2]. Plastic-adherent PBMCs were stored at −80 degrees by freezing container and cultured with serum-free RPMI media in 12-well culture plates (2×10⁶ cells per well) for 20 minutes. The media were replaced with 1 ml of RPMI with 10% FBS with or without LPS for 3 hours Then, media were replaced with 500 μl serum-free RPMI with or without 1 mM CaCl₂) for 60 minutes. The sample supernatants were collected, and the samples were analyzed using IL-1β ELISA kit (BMS224-2, Invitrogen, Carlsbad, CA, USA) by measuring absorbance at 450 nm. Test result was calculated as fold change standardized by one normal control (NC01) and are shown in FIG. 3.

Serum Cytokine Measurement (ELISA Assay)

Levels of IL-1β secretion from the cultured PBMCs were compared among control (NC-01), non-syndromic autoinflammatory hearing loss (DFNA34) and one AIHL subject (AIHL 2) under three conditions: no stimulation, stimulation with LPS, or stimulation with LPS+CaCl₂). IL-1β level upon stimulation with LPS in Case 17-2 was significantly higher than those in NC01 and Case 17-1 (P=0.008 and P=0.016, respectively). Likewise, IL-1β secretion of Case 17-2 in response to LPS+CaCl₂) was higher than that of NC01 (P=0.031 by Kniskal-Wallis test and Bonferroni correction) (FIG. 3).

Overall, according to an embodiment of the present disclosure, in the case of sensorineural hearing loss in which IL-1β is oversecreted compared to normal controls, hearing improved in response to IL-1β antagonist treatment. This demonstrates that a method or kit according to an embodiment of the present disclosure may be used to predict or diagnose whether sensorineural hearing loss is caused by an NLRP3 mutation, or whether a subject's sensorineural hearing loss will respond to treatment with an IL-1β antagonist.

What is claimed is:

1. An information providing method for prediction of therapeutic effect based on etiology of sensorineural hearing loss, comprising:
(a) inducing IL-1β secretion from a sample isolated from a subject;
(b) measuring the IL-1β secretion induced in step (a); and
(c) comparing the IL-1β secretion measured in step (b) to a sample of a normal subject,
wherein the subject is a sensorineural hearing loss patient who has an oversecretion of IL-1β compared to a normal subject, and
wherein, if the subject's hearing level before administration of an IL-1β antagonist is 60 dB or less, the hearing level after administration of the IL-1β antagonist is predicted to be no worse than 60 dB.

2. The method of claim 1, wherein step (a) induces the IL-1β secretion with one or more agents selected from a group consisting of LPS, ATP, and CaCl₂).

3. The method of claim 1, wherein the sample in step (a) is whole blood, peripheral blood mononuclear cells (PBMCs) derived from whole blood, serum, or saliva.

4. The method of claim 1, wherein the measurement of IL-1β secretion in step (b) is performed by enzyme-linked immunosorbent assay (ELISA), RT-PCR, or rapid antigen test.

5. A kit for prediction of therapeutic effect based on etiology of sensorineural hearing loss in a subject comprising an IL-1β secretion-inducing agent and an IL-1β secretion-measuring agent,
wherein the subject is a sensorineural hearing loss patient who has an oversecretion of IL-1β compared to a normal subject, and
wherein, if the subject's hearing level before administration of an IL-1β antagonist is 60 dB or less, the hearing level after administration of the IL-1β antagonist is predicted to be no worse than 60 dB.

6. The kit of claim 5, wherein the IL-1β secretion-inducing agent includes one or more agents selected from a group consisting of LPS, NTP, and CaCl₂.

7. The kit of claim 5, wherein the IL-1β secretion-measuring agent includes one or more agents selected from a group consisting of a primer, a probe, and an antibody.

8. The kit of claim 5, comprising:
a test part that induces IL-1β secretion from a sample isolated from an subject;
a measurement part that measures the induced IL-1β secretion; and
an analyzer that compares the measured IL-1β secretion to a sample of a normal subject.

9. The kit of claim 8, further comprising a display that displays a difference or percentage change in IL-1β secretion before and after inducing IL-1β secretion.

10. The kit of claim 8, further comprising a PBMC isolation extractor.

11. The kit of claim 8, further comprising an autoimmune disease or FTA-ABS testing unit.
